(19) United States
(12) Patent Application Publication
(10) Pub. No.: US 2019/0246915 A1
(43) Pub. Date: Aug. 15, 2019

(54) SYSTEM AND METHOD FOR RECONSTRUCTING A PHYSIOLOGICAL SIGNAL OF AN ARTERY/TISSUE/VEIN DYNAMIC SYSTEM OF AN ORGAN IN A SURFACE SPACE

(71) Applicants: Olea Medical, La Ciotat (FR); Universite d'Aix-Marseille, Marseille (FR); Centre National de la Recherche Scientifique, Paris (FR)

(72) Inventors: Lucie THIEBAUT LONJARET, Marseille (FR); Christine BAKHOUS, Montreal (CA); Olivier COULON, Roquevaire (FR); Sylvain TAKERKART, Marseille (FR)

(73) Assignees: Olea Medical, La Ciotat (FR); Universite d'Aix-Marseille, Marseille (FR); Centre National de la Recherche Scientifique, Paris (FR)

(21) Appl. No.: 16/331,705

(22) PCT Filed: Sep. 8, 2017
(86) PCT No.: PCT/FR2017/052387

§ 371 Date: Mar. 8, 2019

(30) Foreign Application Priority Data
Sep. 9, 2016 (FR) 1658426

(51) Int. Cl. A61B 5/026; A61B 5/055; G06T 7/00

(52) U.S. Cl. CPC A61B 5/0263; G16H 30/40; G06T 7/0012; A61B 5/055

(57) ABSTRACT

The invention concerns a system and method for reconstructing a physiological signal of an artery/tissue/vein system of an organ in a surface space. Said method is implemented by processing means of a processing unit of a functional imaging analysis system, and comprises a step for reconstructing said physiological signal from a piece of experimental data of a region of interest comprising an elementary volume—called a voxel—of said organ and a surface mesh describing said surface space. The invention differs from known methods in particular in terms of the high accuracy of same and the robustness thereof to noise.

The invention relates to a system and method for reconstructing a physiological signal of an artery/tissue/vein dynamic system of an organ in a surface space. Such a method in particular makes it possible to generate an image in the form of a functional activity map. The invention in particular differs from known methods in terms of its high accuracy and its robustness to noise.

According to one preferred but non-limiting exemplary embodiment, the invention will be described as it applies to the brain. However, the invention cannot be limited to this organ alone and may for example be applied to the breast or kidney.

The invention is in particular based on Magnetic Resonance Imaging (also known by the abbreviation “MRI”), more particularly Functional Magnetic Resonance Imaging (fMRI). These techniques make it possible to obtain precious information quickly about the organs of humans or animals. This information is particularly crucial for a practitioner seeking to establish a diagnosis and make a therapeutic decision on the treatment of pathologies. Although preferably used in conjunction with functional Magnetic Resonance Imaging, the invention cannot, however, be limited to this type of imaging or this acquisition protocol alone. To that end, the invention could advantageously be used in any other protocol seeking to study any functional signal, optionally cortical, such as, by way of non-limiting example, positron emission tomography (PET).

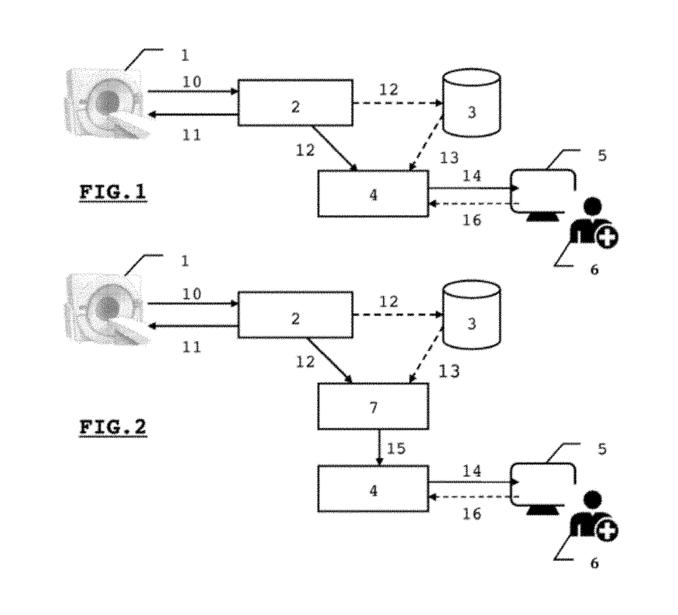

In order to implement such techniques, a Nuclear Magnetic Resonance imaging apparatus 1, as illustrated by way of non-limiting example by FIGS. 1 and 2, is generally used. The latter may deliver multiple sequences of digital images 12 of one or several parts of a patient's body, by way of non-limiting examples, the brain, the heart or the lungs. To that end, said apparatus applies a combination of high-frequency electromagnetic waves on the part of the body in question and measures the signal reemitted by certain atoms, such as, but not limited to, hydrogen for Nuclear Magnetic Resonance imaging. The apparatus thus makes it possible to determine the magnetic properties, and as a result, the chemical composition of the biological tissues and therefore their nature in each elementary volume, which is commonly called a voxel, of the imaged volume. The Nuclear Magnetic Resonance imaging apparatus 1 is controlled using a console 2. The user can thus choose parameters 11 to control the apparatus 1. From information 10 produced by said apparatus 1, multiple digital imaging sequences 12 are obtained of part of a human or animal body.

The sequences of images 12 can optionally be stored on a server 3 and form a medical record 13 of a patient. Such a record 13 can comprise different types of images, such as functional images, showing the activity of the tissues, or anatomical images, reflecting the properties of the tissues. The sequences of images 12 are analyzed using a dedicated processing unit 4. Said processing unit 4 includes means for communicating with the outside world to collect the images. Said communication means further allow the processing unit 4 to deliver in fine, via output means 4 offering a graphic, audio or other rendering, to a user 6 of the analysis system, in particular a practitioner or researcher, an estimate of one or several physiological signals, optionally formatted in the form of content, from images 12 obtained by Magnetic Resonance Imaging, using a suitable man-machine interface. Throughout the document, “output means” refers to any device, used alone or in combination, making it possible to output a representation, for example graphic, audio or the like, of a reconstructed physiological signal, for the user 6 of a Magnetic Resonance imaging analysis system. Such output means 5 may consist, non-exhaustively, of one or several screens, speakers or other man-machine interfaces. Said user 6, optionally a practitioner, of the analysis system can thus confirm or invalidate a diagnosis, decide on a therapeutic action that he deems adequate, deepen research work, etc. Optionally, this user 6 can configure the operation of the processing unit 4 or output means 5, using parameters 16. For example, he can thus define display thresholds or choose the reconstructed signals for which he wishes to have a representation, for example graphic. There is an alternative, described in connection with FIG. 2, for which an imaging system, as previously described, further includes a preprocessing unit 7 for analyzing the sequences of images, deducing experimental signals 15 therefrom and delivering the latter to the processing unit 4, which is thus discharged from this task. Furthermore, to perform a reconstruction of physiological signals, the processing unit 4 generally includes processing means, such as one or several calculators or processors, for carrying out a reconstruction method in the form of a program previously loaded into storage means cooperating with said processing means.

Thus, the acquisition of one or several experimental data, advantageously one or several experimental signals, by Magnetic Resonance Imaging, can be done by regularly sampling a parallelepiped volume in a given slice plane. The obtained two-dimensional images are formed of pixels having a thickness corresponding to the thickness of the slice and called voxels. Such an imaging technique thus makes it possible to acquire both anatomical images, for example to make it possible to reflect the properties of the tissues, and functional images, for example to show the activity of the tissues.

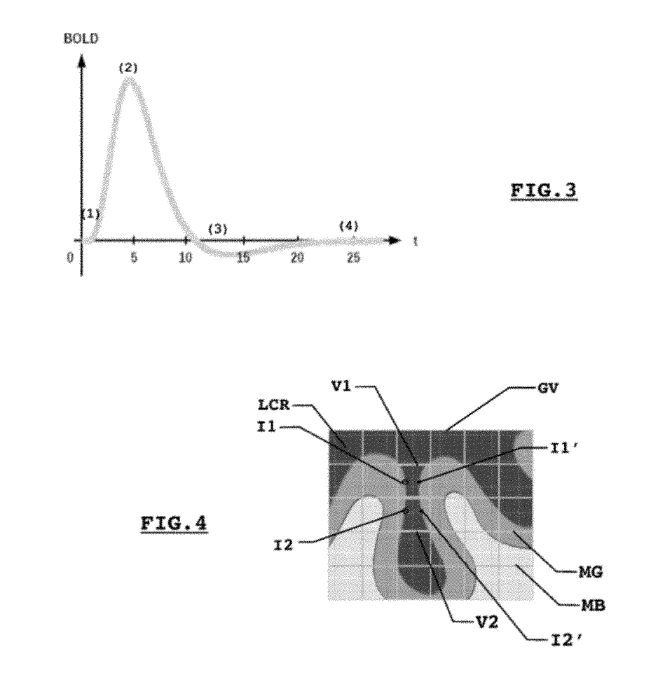

In fMRI, the measurement of the neuronal activity is indirect. Indeed, no apparatus and/or no technique are adapted and/or arranged to guarantee such a measurement. However, studies have demonstrated that the cerebral, more particularly neuronal, activity had a direct impact on blood flow and its composition. Therefore, methods using fMRI may include steps for recording local cerebral hemodynamic variations, namely within the gray matter, when the latter is active, said activation having an impact on the value assumed by the voxel representing said gray matter portion. Thus, it is the changes that such neuronal activity causes in the blood that may ultimately be estimated. The BOLD (Blood Oxygenation Level Dependent) signal, i.e., a signal reflecting the local and temporary variations in the oxygenated hemoglobin concentration in the blood as a function of the neuronal activity of the brain, may then be studied. The study of the BOLD signal is therefore based on the analysis of the oxygenated hemoglobin ratio (also referred to as “oxyhemoglobin”), which has diamagnetic properties relative to the deoxygenated hemoglobin (also known as “deoxyhemoglobin”), which in turn has paramagnetic properties, in the blood. Due to its paramagnetic properties, the deoxygenated hemoglobin causes a decrease in the MRI signal. In reality, when a neuronal zone is activated, the local energy demand, i.e., the demand for added nutrients and oxygen, increases. To meet this increase in demand, the blood flow then increases in such a neuronal zone, much more significantly in comparison, than the oxygen demand. Thus, a decrease in the deoxyhemoglobin concentration, and as a result an increase in the BOLD signal, is observed in the activated zone. The evolutions inherent to such a neuronal activation can be described by a function, the Hemodynamic Response Function (HRS). Such a hemodynamic response function makes it possible to observe the variations of the BOLD signal as a function of time. FIG. 3 shows said hemodynamic response function. Such a function is primarily broken down into four phases. According to FIG. 3, t=0 corresponds to the moment at which the neuron is activated: the BOLD signal first shows a slight decrease (shown on the curve by reference (1)), before increasing sharply until reaching a maximum peak (shown on the curve by reference (2)), around five to six seconds after the activation of the neuron, followed by a new decrease (shown on the curve by reference (3)), until below the baseline around ten to fifteen seconds, then next return to the baseline around twenty-five seconds (situation shown on the curve by reference (4)).

Furthermore, the BOLD signal corresponding to a ratio, there is no absolute scale for the measurement of said BOLD signal. Furthermore, the amplitude of said BOLD signal depends on many factors, such as but not limited to the characteristics inherent to the apparatus and/or the functional imaging system, the acquisition parameters used or the type of tissues passed through, in particular the local water concentration of said tissues. In order to reduce the uncertainties introduced by all of these factors during the observation of the BOLD signal, the percent signal change (also referred to using the abbreviation “PSC”), corresponding to the ratio of the difference between the value of the signal and the baseline value to the baseline value, is generally used to characterize the BOLD signal. In fMRI, the SCP values generally encounter between 0.1% and 5%, making the variations particularly difficult to show on the scale of an individual. Indeed, without an increase of the signal-to-noise ratio, said signal tends to hide the small fluctuations of said BOLD signal. Furthermore, the spatial and temporal resolutions are relatively limited in fMRI when said experimental data volumes are acquired. Indeed, such experimental data volumes then have an approximate precision, then causing a restrictive loss of information.

As a result, after their acquisition, the experimental signals, in the form of data volumes, are generally postprocessed and analyzed in their acquisition space through voxel-based techniques. However, such voxel-based techniques have a certain number of drawbacks. The main drawback of these techniques is granting too little importance and interest to the structural and/or anatomical characteristics of the brain, more generally of the organ to be studied. As a result, the obtained information is not always reliable, since such information relative, for example, to the BOLD cortical signals coming from opposite sides of a sulcus may optionally be “mixed.” Indeed, the low “contrast of the BOLD signal-to-noise” ratio observed in fMRI requires the use in these techniques of a denoising step or a step for enhancing the signal of interest. The most common method for carrying out such a denoising step is a step for filtering acquired experimental data using a low-pass filter, resulting in the production of averaged data from the data contained in the voxels on their direct vicinity. However, the vicinity in the “grid” of the voxels does not always correspond to the actual vicinity in the very convoluted structure of the cortex. FIG. 4 shows an example of a voxel grid representing a sulcus of a cortical surface of a human brain. According to FIG. 4, such a voxel grid GV has thirty voxels, represented in FIG. 4 by thirty squares of identical size. Said voxel grid GV shows a sectional view of a sulcus of the brain. According to FIG. 4, a sulcus is a geometric structure designating the concave folds of the brain, near which three types of tissue are found: white matter MB, gray matter MG and cerebrospinal fluid LCR. Still in the voxel grid GV, two of the voxels V1 and V2 respectively combine the information I1, I1′ and I2, I2′ coming from either side of the sulcus. In the geodesic domain, i.e., along the surface, said information I1, I1′ and I2, I2′ is, however, distinct. In geometry, the shortest path, or one of the shortest paths if there are several, between two points in a space with a metric or distance is a geodesic. Thus, according to FIG. 4, the shortest path to go respectively from information I1, I2 to information I1′, I2′ goes through the bottom of the sulcus. By opposition, in the voxel domain, the information I1, I1′ and I2, I2′ is respectively found in the same voxels V1, V2.

The methods or approaches based on the cortical surface (also referred to as “SBM—Surface-Based Method) can solve this problem by studying the cortical signals in their original surface space, in the case at hand the cortical surface, in order to always take into account the geometry of said original space. Given the anatomical organization of the cortex in functional units (also known as “cortical columns”) perpendicular to the cortical surface, the cerebral microvascular organization in blood vessels globally following said columns, and the acquisition resolution (about three millimeters) close to the thickness of the cortex, it is in fact possible, in the context of BOLD signals, to liken the cortex to a surface. Among all of the known SBM approaches allowing this likening, two approaches and types of methods stand out: those called geometric, and those, more evolved, called anatomically informed.

The geometric methods, which are conceptually very simple, are generally based on interpolation methods, i.e., mathematical operations making it possible to build a curve from a finite number of points, for example, interpolation to the closest neighbor, trilinear interpolation, or a convolution with an ellipsoid filter, or quite simply on the assignment, to each vertex of a surface mesh, of the value of the voxel containing the vertex. Each of these techniques, despite the advantage imparted to them by their simplicity, has a certain number of drawbacks, primarily related to the close relationship between the characteristics inherent to the images and the surface projection method used. Thus, although a geometric approach may, for example, preserve the spatial adjacency relationships, it may also cause the loss of relevant information, such as the size of neuronal activations. Irrespective of the geometric approach used, the latter generally does not observe differences in the information relative to the signals derived from the cerebrospinal fluid (CSF), white matter (WM) and/or the gray matter (GM), which proves to be an aberration relative to the cortical signals, since the neuronal activations are located in the gray matter GM only. Additionally, such geometric methods suffer from a lack of robustness to segmentation errors and/or anatomical-functional recalibration errors.

The anatomically informed methods, based on the principle of surface approaches, in turn try to represent the functional signals in their original space in order to output their initial characteristics in particular taking into account anatomical specificities of the cortex. With this aim, such anatomically informed methods introduce additional information related at least to the anatomy, in some cases to the physiology of the studied organ or the characteristics inherent to the imaging, in particular acquisition modalities, used.

Various researchers, among whom Messrs. Kiebel, Grova, Warnking or even Operto have engaged in this exercise, have developed anatomically informed methods, in particular using methods for re-projecting a physiological signal in a surface space describing the geometry of the domain in which said physiological signal is defined from one or several experimental data acquired by a functional imaging analysis system, taking into account a priori information relative to the anatomy, the physiology of the studied organ or the characteristics inherent to the imaging, in particular acquisition, modalities used. Generally, the term “projection” is defined as the passage from a first space or coordinate system to a second space or coordinate system, using suitable mathematical methods, for example, the method of least squares. Thus, within the meaning of the invention and throughout the document, “reprojection” refers to any change of space or coordinate system done with the aim of re-situating oneself in the space or in the surface coordinate system describing the geometry of the organ from which the physiological signal(s) are derived, in the case at hand, in the context of the preferred exemplary application in connection with the brain, the cortical surface.

The methods currently used also have many drawbacks, quite often leading to a substantial loss of information and potentially the output of an incomplete or even largely irrelevant physiological signal. Indeed, the methods should consider the difference between the resolution of the images acquired by functional Magnetic Resonance Imaging in the form of voxels and the dimension of the cortical columns primarily making up the cortical surface and containing the information relative to the physiological signal: while a voxel generally has dimensions of around several millimeters, more particularly two or three millimeters, a cortical column has dimensions of around one tenth of a millimeter. Lastly, a voxel may then simultaneously contain the information from several columns. Furthermore, the experimental data, also known as “functional volumes,” acquired by functional Magnetic Resonance Imaging, generally corresponds to three-dimensional images. As previously specified, each experimental datum, in the form of a voxel, is associated with a unique experimental signal value. However, a voxel may be located at the border between two tissues, and the signal associated with such a border position then reflects the Magnetic Resonance phenomena of two entities with different tissue properties. Therefore, a voxel may potentially contain signals or information respectively derived from different tissues and mix them indiscriminately.

As previously specified, a technique usable to overcome this drawback may consist of projecting experimental data on the cortical surface using known methods, such as the interpolation approaches. However, such methods suffer from a general lack of robustness to noise and generally do not take account of the temporal dimension. Yet, as already previously stated, the data acquired by functional Magnetic Resonance Imaging, and in fine the physiological signal of interest, are quite often “polluted” by a high noise level. In a region of interest, the presence or absence of functional activity, for example neuronal, is detected by the temporal dimension of the signal. Yet the processing of such a temporal dimension is only done once the surface projection method is carried out and done and the physiological signal estimated, during the implementation of a consecutive second method seeking to detect functional activations. Yet the noise tainting the temporal dimension results from an initial process inherent to the acquisition process of the experimental data by the system. Thus, during the reprojection of the experimental data and/or of the physiological signal, the errors and biases relative to the noise are also propagated during the method. Therefore, the current methods only offer partially effective solutions. Indeed, the information relative to physiological signal re-projected in the surface space thus lacks relevance, since the current methods address the temporal dimension of the experimental data little or not enough.

The invention makes it possible to resolve all or some of the drawbacks raised by the known solutions.

Among the many advantages provided by the invention, we can mention that it makes it possible to:
propose a method allowing the reconstruction of a physiological signal, independently of the characteristics of the active zone of the studied organ;
obtain better results by considerably improving the robustness to noise, both spatially and temporally, thus making it possible to increase the quality of the reconstructed physiological signal and ultimately the detection of functional activations;
increase the robustness of the produced information, despite any recalibration errors between the functional and anatomical data, the uncorrected distortion errors and/or the segmentation errors of the anatomical volume.

To that end, in particular a method is provided for reconstructing a physiological signal of an artery/tissue/vein system of an organ in a surface space, said method being implemented by processing means of a processing unit of a functional imaging analysis system, and comprising a step for reconstructing said physiological signal from an experimental datum of a region of interest comprising an elementary volume—called voxel—of said organ and a surface mesh describing said surface space. According to the invention, the step for reconstructing said physiological signal of such a method consists of evaluating, according to a method for solving an inverse problem, an a posteriori marginal distribution for said physiological signal in a vertex of said mesh by:
assigning a direct probability distribution of the experimental datum in said surface space knowing the parameters involved in the reconstruction problem of the physiological signal of the artery/tissue/vein dynamic system for the voxel in question;
jointly assigning an a priori spatial probability distribution of said physiological signal by introducing a priori information relative to a characteristic of the experimental datum and/or a priori information relative to a property of the artery/tissue/vein dynamic system;
jointly assigning an a priori temporal probability distribution of said physiological signal by introducing a priori information relative to the impulse response of said artery/tissue/vein dynamic system.

The invention further provides a method for reconstructing a physiological signal of an artery/tissue/vein dynamic system of an organ in a surface space, said method being implemented by processing means of a processing unit of a functional imaging analysis system, and comprising a step for reconstructing the physiological system from an experimental datum of a region of interest comprising an elementary volume—called voxel—and a surface mesh describing said surface space. According to the invention, and like before, the step for reconstructing said physiological signal of such a method consists of evaluating, according to a method for solving an inverse problem, a cost function for said physiological signal in a vertex of said mesh by:
assigning an operator of the direct model establishing the link between the experimental datum and the elementary volume and said physiological signal in said surface space knowing the parameters involved in the problem of the reconstruction of the physiological signal of the artery/tissue/vein dynamic system for the voxel in question;
jointly assigning a spatial regularization operator by introducing a priori information relative to a characteristic of the experimental datum and/or a priori information relative to a property of the artery/tissue/vein dynamic system;
jointly assigning a temporal regularization operator by introducing a priori information relative to the impulse response of said artery/tissue/vein dynamic system.

To allow quick and particularly effective diagnoses, as well as brief exams, a method for reconstructing a physiological signal according to the invention may further comprise a step for producing said experimental datum from an acquisition of a signal by functional imaging.

Advantageously, when the functional imaging analysis system comprises output means for the reconstructed physiological signal for a user of said system, said output means cooperating with the processing unit, a method according to the invention may comprise a subsequent step for triggering a output of the reconstructed physiological signal in an appropriate format.

To improve the quality of the experimental signals obtained and acquired by functional imaging and ultimately the quality of the obtained results, a method according to the invention may further comprise a prior step for preprocessing of the experimental datum and/or the surface mesh, said step being arranged to correct and/or recalibrate the experimental datum and/or the surface mesh, respectively.

Advantageously, when the functional imaging analysis system comprises output means for a user of said system, said output means cooperating with the processing unit, a method according to the invention may further comprise a subsequent step for triggering the output of the reconstructed physiological signal in one or several vertices of the mesh for each voxel of the region of interest and generating an image in the form of a functional activity map.

According to a second object, the invention relates to a processing unit comprising means for communicating with the outside world and processing means, cooperating with storage means. Advantageously, the communication means are arranged to receive, from the outside world, an experimental datum, and the storage means comprise instructions executable or interpretable by the processing means, the interpretation or execution of said instructions by said processing means causing the implementation of a method according to the first or second object of the invention.

To help a practitioner seeking to establish a diagnosis, the communication means of a processing unit according to the invention can deliver a reconstructed physiological signal in an appropriate format to output means suitable for retrieving it for a user.

According to a third object, the invention relates to a functional imaging analysis system comprising a processing unit according to the invention and output means able to output, for a user, a physiological signal according to a method according to the first object of the invention and implemented by said processing unit.

Lastly, according to a fourth object, the invention relates to a computer program product comprising one or several instructions interpretable or executable by the processing means of a processing unit according to the invention. Said processing unit further comprises storage means or cooperating with such storage means, said program being loadable in said storage means. Said instructions by said storage means are such that their interpretation or execution causes the implementation of a method according to the first or second object of the invention.

Other features and advantages will appear more clearly upon reading the following description and examining the figures that accompany it, in which:

FIGS. 1 and 2, previously described, present two alternative embodiments of a medical imaging analysis system, optionally by Magnetic Resonance;

FIG. 3, previously described, shows an example of a hemodynamic response function;

FIG. 4, previously described, shows an example of a partial voxel grid showing a sulcus of a cortical surface of the human brain;

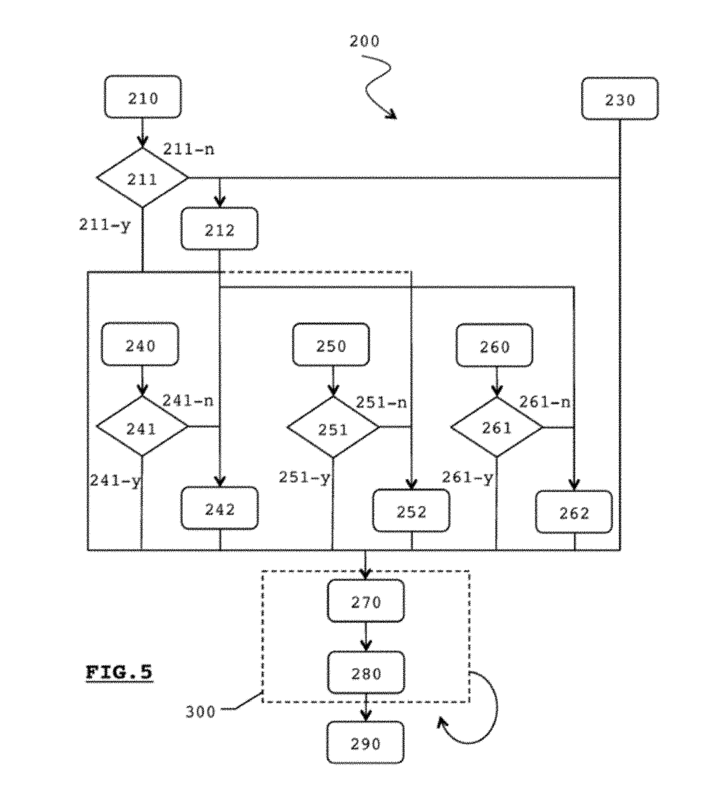

FIG. 5 schematically describes a simplified flowchart of a method according to the invention;

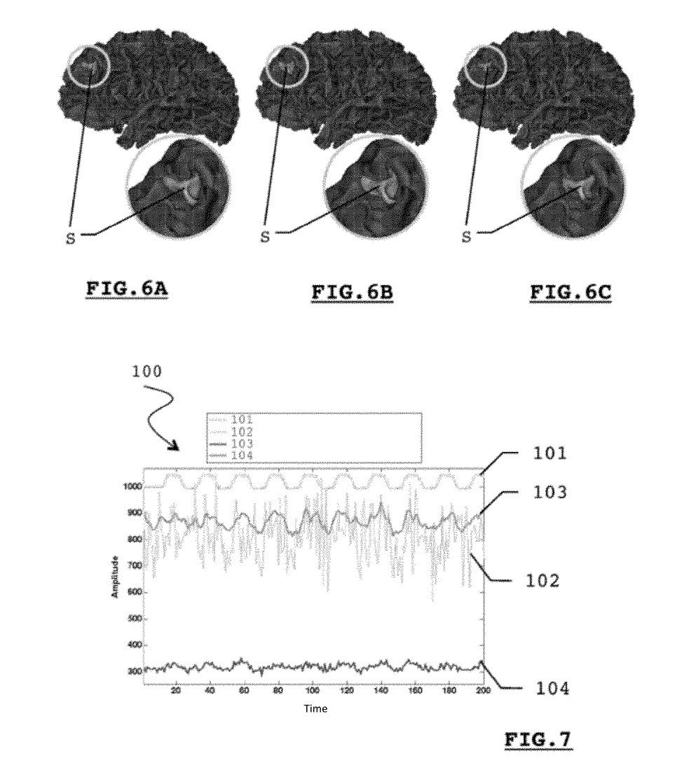

FIGS. 6A, 6B and 6C show three examples of static textures, respectively generated and output using a method according to the invention, corresponding to the original or reference signal, generated and output using to a method according to the State of the Art;

FIG. 7 shows a set of four examples of time courses of a physiological signal at a same vertex, respectively an original or reference physiological signal, a noised physiological signal of a voxel containing said vertex, a physiological signal re-projected by a method according to the invention and a physiological signal reconstructed by a method according to the State of the Art;

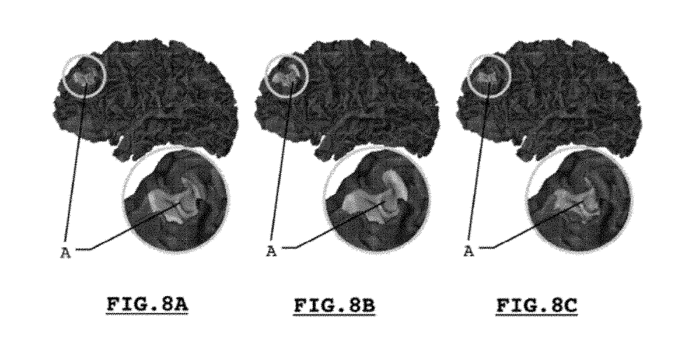

FIGS. 8A, 8B and 8C show three examples of functional activity maps, respectively generated and output from a physiological signal respectively reconstructed using a method according to the invention, corresponding to the original or reference signal and re-projected using a method according to the State of the Art.

FIG. 5 schematically shows a method 200 for reconstructing a physiological signal of an artery/tissue/vein dynamic system of an organ in a surface space according to the invention. As previously specified, such a method 200 is advantageously implemented by processing means of a processing unit 4 of a Magnetic Resonance imaging analysis system or more generally a functional imaging analysis system, such as, by way of non-limiting examples, those described in connection with FIGS. 1 and 2. Within the meaning of the invention and throughout the entire document, “reconstruction of a physiological signal” refers to the generation of a physiological signal from one or several experimental data previously acquired by functional imaging. Additionally, within the meaning of the invention and throughout the entire document, a “surface space” is defined as a space describing the geometry of the artery/tissue/vein dynamic system of an organ of interest. In the context of the preferred exemplary application described in connection with the brain, such as surface space, also called “cortical surface,” consists of a surface taken in the cortical ribbon, also known as the “cortex.” A method 200 according to the invention advantageously comprises a step 300 for reconstructing said physiological signal from an experimental datum of an elementary volume—called voxel—of said organ and a surface mesh describing said surface space.

As a reminder, within the meaning of the invention and throughout the document, “voxel” (contraction of the term “volumetric pixel”) refers to an elementary volume making it possible to measure the definition of a bitmap digital image in three dimensions. Such a voxel may also be considered a three-dimensional pixel. In all cases, such a voxel may be considered a parallelepiped rectangle whereof the closed surface is formed of its six faces. Additionally, within the meaning of the invention and throughout the document, “surface mesh” refers to any geometric modeling of said surface space preferably by finite and well-defined proportioned elements. Alternatively, such a surface mesh may consist of the geometric modeling of said surface space by parameterized surfaces or implicit surfaces, for example mathematical functions known as “level-set.” Thus, as a preferred but non-limiting example, a “surface mesh” is defined as a three-dimensional (3D) network formed of vertices connected to one another by edges, i.e., three-dimensional segments delimited by two vertices, and thus forming a set of faces. In the context of our preferred but nonlimiting example and in connection with the brain, said vertices may advantageously consist of points of the three-dimensional space located on, in or near the cortical ribbon.

A method 200 according to the invention comprises a processing operation 300 in order to reconstruct a physiological signal primarily consisting of a step 270 in order to assign and/or evaluate one or several a posteriori marginal distributions for said physiological signal that one is seeking to reconstruct, such as the BOLD signal in a vertex of the mesh. Such a processing operation 300 further comprises a step 280 in order to calculate the value of said signal strictly speaking. To evaluate such an a posteriori marginal distribution, it is necessary to configure, manually or automatically, the processing unit 4 of a functional imaging analysis system, like that previously described in connection with FIGS. 1 and 2. This configuration can preferably be done by the processing unit 4 itself, owing to its processing means, from one or several configuration parameters. The configuration can also consist in the formation of a library of one or several a posteriori marginal distributions, the library being preestablished and stored in a memory of programs and/or a data memory, commonly called storage means, of said unit. The invention provides that said library can be extended as it is used, or even output by an external computing unit able to perform said configuration from the configuration parameter(s) and able to cooperate with the processing unit to deliver said library.

A method 200 according to the invention may thus comprise configuration steps 240, 250, 260 carried out prior to the assignment 270, manually or automatically, among which the following are necessary and sufficient:
assigning 240 the direct probability distribution of the experimental data in said surface space knowing the parameters involved in the reconstruction problem of the physiological signal of the artery/tissue/vein dynamic system for the voxel in question;
assigning 250 an a priori spatial probability distribution of said physiological signal by introducing a priori information relative to one or several characteristics of the experimental data and/or a priori information relative to one or several properties of the artery/tissue/vein dynamic system;
assigning 260 an a priori temporal probability distribution of said physiological signal by introducing a priori information relative to the impulse response of said artery/tissue/vein dynamic system.

Within the meaning of the invention and throughout the entire document, the term “direct probability distribution” may advantageously be qualified as “likelihood function.”

Furthermore, the invention provides that a method 200 for reconstructing a physiological signal according to the invention can also comprise a configuration step 210 arranged to allow the assignment of a surface mesh describing the surface space of the studied organ.

The configuration steps can depend on the considered application area. Additionally, before the configuration steps 210, 240, 250, 260, a method 200 for reconstructing a physiological signal according to the invention may advantageously and respectively comprise test steps 211, 241, 251, 261 for verifying the specification by the user of:
the assignment 210 of a surface mesh describing the surface space of the studied organ;
the assignment 240 of the direct probability distribution of the experimental data in said surface space knowing the parameters involved in the reconstruction problem of the physiological signal of the artery/tissue/vein dynamic system for the voxel in question;
the assignment 250 of an a priori spatial probability distribution of said physiological signal by the introduction of a priori information relative to one or several characteristics of the experimental data and/or a priori information relative to one or several properties of the artery/tissue/vein dynamic system;
assigning 260 an a priori temporal probability distribution of said physiological signal by introducing a priori information relative to the impulse response of said artery/tissue/vein dynamic system.

Such test steps or operations 211, 241, 251, 261 can advantageously consist of testing the value of a Boolean indicator initialized or updated by one or several configuration or customization steps, previously mentioned, of the functional imaging analysis system of which the processing unit implements a method to reconstruct a physiological signal according to the invention or any other technique implemented by the processing unit capable of guaranteeing such a configuration or such a customization of the functional imaging analysis system.

If all of the assignments have been configured beforehand (situations symbolized by references 211-y, 241-y, 251-y, 261-y in FIG. 5), the processing unit implements the subsequent steps of a method 200 according to the invention. Otherwise, if no direct probability distribution of the experimental data has been configured (situation symbolized by reference 241-n in FIG. 5) beforehand, a method 200 according to the invention comprises a step 242 for constructing said direct probability distribution from a priori information relative for example, but not limited to, the anatomy of the surface space comprising the experimental datum or data, the nature of the physiological signal to be reconstructed, the acquisition parameters of the experimental datum or data, etc. Such a construction can preferably be done by the processing unit 4 itself, using its processing means, from one or several construction parameters. The construction can also consist in the formation of a library of one or several direct probability distributions, the library being preestablished and stored in a program memory of said unit, one of said direct probability distributions of which can be selected. Similarly, if no a priori spatial or a priori temporal probability distribution of said physiological signal has been configured (situations symbolized by references 251-n, 261-n in FIG. 5) beforehand, a method 200 according to the invention respectively comprises steps 252, 262 for constructing an a priori spatial probability distribution and an a priori temporal probability distribution. Similarly, if no surface mesh has been configured (situation symbolized by reference 211-n in FIG. 5) beforehand, a method 200 according to the invention comprises a step 212 for constructing a surface mesh describing said surface space from anatomical information relative to the studied organ. Alternatively or additionally, if no a posteriori marginal distribution for said physiological signal has been configured or assigned (situation not shown in FIG. 5) beforehand, the invention provides that a method according to the invention may comprise a step for constructing said a priori marginal distribution.

The described prior steps of a method for reconstructing a physiological signal according to the invention have been described in connection with a probabilistic approach, but remain relevant for the implementation of a deterministic approach. Thus, step 270 for evaluating one or several a posteriori marginal distributions for said physiological signal may consist, according to a deterministic approach, of a step 270 for evaluating one or several cost functions for reconstructing said physiological signal. Likewise, the configuration steps 240, 250, 260 implemented before the assignment 270, manually or automatically, may consist, according to a deterministic approach, of:
assigning 240 the operator of the direct model establishing the link between the experimental datum in the elementary volume and said physiological signal in said surface space knowing the parameters involved in the reconstruction problem of the physiological signal of the artery/tissue/vein dynamic system for the considered voxel;
assigning 250 a spatial regularization operator by introducing a priori information relative to a characteristic of the experimental datum and/or a priori information relative to one or several properties of the artery/tissue/vein dynamic system;
assigning 260 a temporal regularization operator by introducing a priori information relative to the impulse response of said artery/tissue/vein dynamic system.

Similarly to the direct probability distribution, if no operator of the direct model of the experimental data has been configured (situation symbolized by reference 241-n in FIG. 5) beforehand, a method 200 according to the invention comprises a step 242 for constructing said operator of the direct model from a priori information relative to, for example but not limited to, the anatomy of the surface space comprising the experimental datum or data, the nature of the physiological signal to be reconstructed, the acquisition parameters of the experimental datum or data, etc. Such a construction may be done preferably by the processing unit 4 itself, using its processing means, from one or several construction parameters. The construction may also consist in the formation of a library of one or several operators of the direct model, the library being preestablished and stored in a program memory and/or a data memory, commonly called storage means, of said unit, one of said operators of which of said direct model may be selected. Similarly, if no a priori spatial or temporal regularization operator of said physiological signal has been configured (situations symbolized by references 251-n, 261-n in FIG. 5) beforehand, a method 200 according to the invention respectively comprises steps 252, 262 for constructing a spatial regularization operator or a temporal regularization operator.

First and second exemplary implementations of such a method 200 for reconstructing a physiological signal, respectively according to deterministic and probabilistic approaches, will advantageously but non-limitingly be described in the remainder of the document, in connection with FIG. 5 making it possible to illustrate a method 200 for a physiological signal according to the invention. To that end, an experimental datum, in the form of a functional volume V, is acquired at each moment t.

According to a first exemplary implementation according to a deterministic approach, an experimental direct model was chosen and defined on the one hand to reflect the physiological behavior of the BOLD signal, in particular exposing the propagation of a neuronal activity of a cortical column to its adjacent columns such that: at each moment t, a cortical physiological signal A at a vertex n of a surface mesh influences its adjacent vertices m of said surface mesh according to a weight ω_{geodesic}(n,m) inversely proportional to the geodesic distance, i.e., along the surface, separating them. Furthermore, said experimental direct model has been chosen and defined on the other hand to model the physical phenomena in play during the acquisition of one or several experimental data by a Magnetic Resonance imaging system, in particular describing the partial volume effect, such that: at each moment t, a cortical physiological signal A in a vertex n of a surface mesh influences the voxels ν surrounding said vertex n, i.e., normally on the surface, according to a maximum weight ωₙₒᵣₘₐₗ(ν,n) in the gray matter and inversely proportional to the distance between said voxels and the gray matter once said voxels are positioned in the white matter and the cerebrospinal fluid. Therefore, a direct model, in the form of a normal weight and geodesic model, can be written in the form of the following system of equations:

$$\{ \begin{matrix}V ( v , t ) = {\sum }_{n = 1}^{{N}_{n}} {ω}_{normal} ( v , n ) A ( n , t ) \\ A ( n , t ) = {\sum }_{m = 1}^{{N}_{n}} {ω}_{geodesic} ( n , m ) A ( m , t )\end{matrix} ⇒ V ( v , t ) = {\sum }_{m = 1}^{{N}_{n}} M ( v , m ) A ( m , t )$$

Thus, a method according to the invention comprises a configuration step 240 for assigning an operator of the direct model M in the form of a size matrix N_{ν}×Nₙ, where N_{ν} is the number of voxels v contained in an experimental datum V, also known as functional volume, and Nₙ is the number of vertices of the surface mesh, establishing the link between the experimental datum V in the elementary volume ν and said physiological signal A in said surface space, knowing the weights ω_{geodesic}(n,m) and ωₙₒᵣₘₐₗ(ν,n).

Furthermore, a method 200 according to the invention comprises a configuration step 250 for assigning a spatial regularization operator Eₛₚₐₜᵢₐₗ(A) of the physiological signal to be reconstructed by introducing a priori information relative to a characteristic of the experimental datum and/or a priori information relative to a property of the artery/tissue/vein dynamic system, such as:

Eₛₚₐₜᵢₐₗ(A)=λ_{D}Tr((DA)^{t}(DA))

where λ_{D} is a spatial regularization coefficient,
A is the matrix of the physiological signal to be reconstructed,
and D is the spatial regularization matrix written in the form:

$${D}_{i , j} = \frac{{δ}_{j , {c}_{i 1}} - {δ}_{j , {c}_{i 2}}}{{d}_{g} ( {n}_{{c}_{i 1}} , {n}_{{c}_{i 2}} ) {D}_{norm} ( {n}_{{c}_{i 1}} , {n}_{{c}_{i 2}} )} with \\ \vert \begin{matrix}{δ}_{i , j} = \{ \begin{matrix}1 , if i = j \\ 0 , otherwise\end{matrix} \\ {D}_{norm} ( {n}_{{c}_{i 1}} , {n}_{{c}_{i 2}} ) = \sqrt{\frac{\# v ( {n}_{{c}_{i 1}} ) \# v ( {n}_{{c}_{i 2}} )}{\# v ( {n}_{{c}_{i 1}} ) + \# v ( {n}_{{c}_{i 2}} )}}\end{matrix}$$

where δ_{i,j} is the Kronecker symbol, d_{g}(n_{c}_{ᵢ₁},n_{c}_{ᵢ₂}) is the geodesic distance between two vertices at the end of an edge of the surface mesh, Dₙₒᵣₘ(n_{c}_{ᵢ₁},n_{c}_{ᵢ₂}) is a normalization term with n_{c}_{ᵢ₁} and n_{c}_{ᵢ₂} corresponding to the two vertices at the end of an edge cᵢ of the surface mesh and #ν(n_{c}_{ᵢ₁}) and #ν(n_{c}_{ᵢ₂}) respectively corresponding to the number of direct neighbors of the vertices n_{c}_{ᵢ₁} and n_{c}_{ᵢ₂}.

Furthermore, a method 200 according to the invention comprises a configuration step 260 for assigning a temporal regularization operator Eₜₑₘₚₒᵣₐₗ(A) by introducing a priori information relative to the impulse response of said artery/tissue/vein dynamic system, such that:

Eₜₑₘₚₒᵣₐₗ(A)=λ_{T}Tr(AT^{t}TA^{t})

where λ_{T} is a temporal regularization coefficient,
A is the matrix of the physiological signal to be reconstructed,
and T is the temporal regularization matrix written in the form:

$${T}_{order 2} = \frac{1}{{Δ}_{T}^{2}} ( \begin{matrix}1 & - 2 & 1 & 0 & … & 0 \\ 0 & 1 & - 2 & 1 & ⋱ & ⋮ \\ ⋮ & ⋱ & ⋱ & ⋱ & ⋱ & 0 \\ 0 & … & 0 & 1 & - 2 & 1\end{matrix} )$$

where Δ_{T} is a time interval between two acquisitions of two functional volumes.

Furthermore, a method 200 according to the invention includes a configuration step 270 for assigning a cost function E(V,A) for said physiological signal A in a vertex of said mesh, such that:

$$E ( V , A ) = \frac{Tr ( {( V - MA )}^{t} {R}^{- 1} ( V - MA ) )}{2} + {λ}_{D} Tr ( {( DA )}^{t} ( DA ) ) + {λ}_{T} Tr ( {AT}^{t} {TA}^{t} )$$

where

$${E}_{attachment} ( V , A ) = \frac{Tr ( {( V - MA )}^{t} {R}^{- 1} ( V - MA ) )}{2}$$

is the likelihood term to the data measuring the deviation between the direct model applied to the physiological signal A and the experimental data V considering the hypothesis of a Gaussian noise, with M the operator of the direct model and R⁻¹ the covariance matrix of the noise;
Eₛₚₐₜᵢₐₗ(A)=λ_{D}Tr((DA)^{t}(DA)) is the spatial regularization operator previously configured,
Eₜₑₘₚₒᵣₐₗ(A)=λ_{T}Tr(AT^{t}TA^{t}) is the temporal regularization operator previously configured.

Lastly, for optimization purposes, a method 200 according to the invention includes a step 280 for evaluating said cost function E(V,A) for said physiological signal A at a vertex of said mesh. Such a step 280 for evaluating said cost function E(V,A) consists of minimizing such a cost function E(V,A) according to the physiological signal A, while minimizing, or even canceling, the total energy gradient, consisting of solving the following equation:

∇E(V,A)=(M^{t}R⁻¹M+2λ_{D}D^{t}D)A+2λ_{T}AT^{t}T−M^{t}R⁻¹V=0

Lastly, step 280 for evaluating said cost function E(V,A) consists of solving the following system, by the implementation by processing means of a processing unit 4 of a functional imaging analysis system, like that described in connection with FIGS. 1 and/or 2, of the linear conjugated gradients algorithm:

$$HA + AG = K with \{ \begin{matrix}H = {M}^{t} {R}^{- 1} M + 2 {λ}_{D} {D}^{t} D \\ G = 2 {λ }_{T} {T}^{t} T \\ K = {M}^{t} {R}^{- 1} V\end{matrix}$$

According to a second exemplary implementation according to a probabilistic approach, an experimental direct model was chosen and defined on the one hand to reflect the physiological behavior of the BOLD signal, by in particular formulating the propagation of a neuronal activity of a cortical column to its adjacent columns such that, at each moment t, a cortical physiological signal A in a vertex n of a surface mesh influences its adjacent vertices m of said surface mesh according to a weight ω_{geodesic}(n,m) inversely proportional to the geodesic distance, i.e., along the surface, separating them. Furthermore, said experimental direct model was chosen and defined on the other hand to model the physical phenomena in play during the acquisition of one or several experimental data by a Magnetic Resonance imaging system, in particular by describing the partial volume effect, such that: at each moment t, a cortical physiological signal A in a vertex n of the surface mesh influences the voxels ν surrounding said vertex n, i.e., normally on the surface, according to a maximum weight ωₙₒᵣₘₐₗ(ν,n) in the gray matter and inversely proportional to the distance between said voxels and the gray matter once said voxels are positioned in the white matter and the cerebrospinal fluid. Therefore, a direct model, in the form of a normal weight and geodesic model, can be written in the form of the following system of equations:

$$\{ \begin{matrix}V ( v , t ) = {\sum }_{n = 1}^{{N}_{n}} {ω}_{normal} ( v , n ) A ( n , t ) \\ A ( n , t ) = {\sum }_{m = 1}^{{N}_{n}} {ω}_{geodesic} ( n , m ) A ( m , t )\end{matrix} ⇒ V ( v , t ) = {\sum }_{m = 1}^{{N}_{n}} M ( v , m ) A ( m , t )$$

Thus, a method according to the invention comprises a configuration step 240 for assigning a probability distribution M, in the form of a matrix measuring N_{ν}×Nₙ, where N_{ν} is the number of voxels V contained in an experimental datum V, also known as functional volume, and Nₙ is the total number of vertices of the surface mesh, of the experimental datum V and said surface space, knowing the weights ω_{geodesic}(n,m) and ωₙₒᵣₘₐₗ(ν,n) for the considered voxel ν.

Furthermore, a method 200 according to the invention comprises a configuration step 250 for assigning an a priori spatial probability distribution of said physiological signal pₛₚₐₜᵢₐₗ(A) by introducing a priori information relative to a characteristic of the experimental datum and/or a priori information relative to a property of the artery/tissue/vein dynamic system, such as:

$${p}_{spatial} ( A ) = \frac{1}{{Z}_{spatial}} {e}^{- {λ}_{D} Tr ( {( DA )}^{t} ( DA ) )}$$

where λ_{D} is a spatial regularization coefficient,

$$\frac{1}{{Z}_{spatial}}$$

is a normalization term, A is the matrix of the physiological signal to be reconstructed, and D is the spatial regularization matrix written in the following form:

$${D}_{i , j} = \frac{{δ}_{j , {c}_{i 1}} - {δ}_{j , {c}_{i 2}}}{{d}_{g} ( {n}_{{c}_{i 1}} , {n}_{{c}_{i 2}} ) {D}_{norm} ( {n}_{{c}_{i 1}} , {n}_{{c}_{i 2}} )} with \\ \vert \begin{matrix}{δ}_{i , j} = \{ \begin{matrix}1 , if i = j & if \\ 0 , otherwise & \end{matrix} \\ {D}_{norm} ( {n}_{{c}_{i 1}} , {n}_{{c}_{i 2}} ) = \sqrt{\frac{\# v ( {n}_{{c}_{i 1}} ) \# v ( {n}_{{c}_{i 2}} )}{\# v ( {n}_{{c}_{i 1}} ) + \# v ( {n}_{{c}_{i 2}} )}}\end{matrix}$$

where δ_{i,j} is the Kronecker symbol, d_{g}(n_{c}_{ᵢ₁},n_{c}_{ᵢ₂}) is the geodesic distance betwwen two vertices at the ends of an edge of the surface mesh, Dₙₒᵣₘ(n_{c}_{ᵢ₁},n_{c}_{ᵢ₂}) is a spatial regularization term A with n_{c}_{ᵢ₁} and n_{c}_{ᵢ₂} corresponding to the two vertices at the end of an edge cᵢ of the surface mesh and #ν(n_{c}_{ᵢ₁}) and #ν(n_{c}_{ᵢ₂}) respectively corresponding to the number of direct neighbors of the vertices n_{c}_{ᵢ₁} and n_{c}_{ᵢ₂}.

Furthermore, a method 200 according to the invention comprises a configuration step 260 for assigning an a priori temporal probability distribution of said physiological signal pₜₑₘₚₒᵣₐₗ(A) by introducing a priori information relative to the impulse response of said artery/tissue/vein dynamic system, such that:

$${p}_{temporal} ( A ) = \frac{1}{{Z}_{temporal}} {e}^{- {λ}_{T} Tr ( {AT}^{′} {TA}^{′} )}$$

where λ_{T} is a temporal regularization coefficient,

$$\frac{1}{{Z}_{temporal}}$$

is a normalization term, A is the matrix of the physiological signal to be reconstructed, and T is the temporal regularization matrix written in the form:

$${T}_{order 2} = \frac{1}{{Δ}_{T}^{2}} ( \begin{matrix}1 & - 2 & 1 & 0 & … & 0 \\ 0 & 1 & - 2 & 1 & ⋱ & ⋮ \\ ⋮ & ⋱ & ⋱ & ⋱ & ⋱ & 0 \\ 0 & … & 0 & 1 & - 2 & 1\end{matrix} )$$

where Δ_{T} is a time interval between two acquisitions of two functional volumes.

Furthermore, a method 200 according the invention comprises a configuration step 270 for assigning an a posteriori marginal distribution p(A|V) for said physiological signal A in a vertex of said mesh, such that:

$$p ( A \vert V ) ∝ {e}^{- ( \frac{{Tr ( V - MA )}^{t} {R}^{- 1} ( V - MA ) )}{2} + {λ}_{D} Tr ( {( DA )}^{t} ( DA ) ) + {λ}_{T} Tr ( {AT}^{t} {TA}^{t} ) )}$$

where

$${e}^{- ( \frac{{Tr ( V - MA )}^{t} {R}^{- 1} ( V - MA ) )}{2} )}$$

corresponds to the likelihood function, e^{-(λ}^{_{D}}^{Tr((DA)}^{^{t}}^{(DA))} corresponds to the a priori spatial probability distribution, and e^{-(λ}^{_{T}}^{Tr(AT}^{^{t}}^{TA}^{^{t}}⁾⁾ corresponds to the a priori temporal probability distribution, said likelihood, a priori spatial probability distribution and a priori temporal probability distribution functions previously assigned to within a multiplicative constant.

Lastly, for optimization purposes, a method 200 according to the invention comprises a step 280 for evaluating the a posteriori marginal distribution p(A|V) for said physiological signal A in a vertex of said mesh. Such a step 280 for evaluating said a posteriori marginal distribution p(A|V) consists of maximizing the a posteriori marginal distribution p(A|V)according to the physiological signal A, by applying the A Posteriori Maximum Estimator, such that:

$${arg max}_{A} p ( A \vert V ) ) = {arg max}_{A} ( {e}^{- ( \frac{{Tr ( V - MA )}^{t} {R}^{- 1} ( V - MA ) )}{2} + {λ}_{D} Tr ( {( DA )}^{t} ( DA ) ) + {λ}_{T} Tr ( {AT}^{t} {TA}^{t} )} ) ) \\ or \text{:} \\ {arg max}_{A} p ( A \vert V ) ) = arg {min}_{A} ( \frac{{Tr ( V - MA )}^{t} {R}^{- 1} ( V - MA )}{2} + {λ}_{D} Tr ( {( DA )}^{t} ( DA ) ) + {λ}_{T} Tr ( {AT}^{t} {TA}^{t} )$$

Lastly, step 280 for evaluating the a posteriori marginal distribution p(A|V) consists of solving the following system, by the implementation by processing means of a processing unit 4 of a functional imaging analysis system, like that described in connection with FIGS. 1 and/or 2, of the linear conjugated gradients algorithm:

$$HA + AG = K with \{ \begin{matrix}H = {M}^{t} {R}^{- 1} M + 2 {λ}_{D} {D}^{t} D \\ G = 2 {λ }_{T} {T}^{t} T \\ K = {M}^{t} {R}^{- 1} V\end{matrix}$$

Furthermore, a method 200 for reconstructing a physiological signal may comprise a step 230 for producing said experimental datum from an acquisition of a signal by functional imaging. The acquisition of one or several experimental data, advantageously signals, by functional imaging, more particularly Magnetic Resonance Imaging, can be done by regularly sampling a parallelepiped volume in a given slice plane. The experimental data, also known as “images,” obtained in two dimensions are formed of pixels having a thickness, corresponding to the thickness of the slice and called voxels. In MRI, more particularly in fMRI, such an acquisition can be done using one or several sequences defined by acquisition parameters such as, for example, the echo time TE, the repetition time TR, the tilt angle α or the inversion time TI. As a preferred but non-limiting example, a planar echo acquisition sequence (also known as “echo planar imaging” or “EPI”) can be used. Alternatively, a gradient echo sequence, generally provided in all imaging systems, can also be used.

Furthermore, as previously specified, in particular in the example described in connection with FIGS. 1 and 2, a functional imaging analysis system according to the invention may comprise output means 5 of the reconstructed physiological signal for a user 6 of said system, said output means 5 cooperating with the processing unit 4. Such output means 5 make it possible to have a rendering, advantageously graphic, audio or the like, and may for example comprise a screen or speakers. Thus, alternatively or additionally, a method 200 according to the invention may advantageously comprise a subsequent step 290 for triggering a output of the reconstructed physiological signal in an appropriate format. According to the preferred but nonlimiting exemplary application in connection with the brain, the reconstructed physiological signal is advantageously the BOLD signal. For example, the reconstructed physiological signal(s) may advantageously take the form of one or several dynamic surface data. Indeed, for each vertex of the surface mesh in which a physiological signal is reconstructed, at each moment, i.e., for each of the moments corresponding to an experimental datum, the output means of an analysis signal by functional imaging, such as, for example, that previously described in connection with FIGS. 1 and 2, can output a value corresponding to the value of said physiological signal in that vertex and at that moment. Thus, said value(s) of said physiological signal can be output by the output means of said functional imaging analysis system in different forms, in particular one or several graphic representations of different natures, such as, by way of non-limiting examples, one or several time courses, one or several dynamic or static textures.

Within the meaning of the invention and throughout the entire document, “time course” refers to the evolution of a physiological signal over time at a predetermined point, such as a vertex or a voxel of interest, represented by an amplitude curve as a function of time. FIG. 7 in particular shows a set 100 of several examples of such time courses 101, 102, 103, 104 in a same vertex. The curve 101 in particular illustrates the time course of an original or reference physiological signal in a vertex. The curve 102 in turn shows a graphic representation of the time course of the same noised physiological signal of a voxel containing said vertex. In turn, the curve 103 in particular illustrates the time course of a physiological signal reconstructed by a method according to the invention in the same vertex. Lastly, the curve 104 shows a graphic representation of the time course of the physiological signal reconstructed by a method according to the State of the Art in the same vertex. As shown by the example described in connection with the curve 102 of FIG. 7, from experimental data acquired by fMRI, the user cannot extract any relevant information relative to the physiological signal “with the naked eye,” primarily due to the presence of significant noise. The variations of interest of the signal being very small, a noise level, even low, suffices to interfere with the signal and prevents obtaining relevant information relative to the physiological signal. As shown by the example described in connection with the curve 103 of FIG. 7, a method according to the invention makes it possible not only to improve the quality of a reconstructed physiological signal in comparison with the output of the same physiological signal reconstructed by a method according to the State of the Art, as attested by the curve 104 of FIG. 7, but also offers the user the possibility of reading, without additional processing, relevant information contained within said reconstructed physiological signal. For example, by reducing the noise contained in the experimental data, such a method could allow the user, by displaying on the one hand the experimental paradigm convoluted to the hemodynamic response function 101, and on the other hand the time course of the physiological signal reconstructed by said method in different vertices of interest of the surface mesh, to detect correlation relationships between said reconstructed physiological signal and the experimental paradigm convoluted to the hemodynamic response function. As attested by FIG. 7, the time course of the physiological signal reconstructed by a method according to the invention, shown by curve 103 of FIG. 7, is much “cleaner” than the time course of the experimental physiological signal, shown by the curve 102 of said FIG. 7, since the method according to the invention makes it possible to provide a spatial correlation between the physiological signals, while it makes it possible to decrease the impact of the noise and improve the visibility of the variations of interest of said reconstructed physiological signal.

Additionally, within the meaning of the invention and throughout the entire document, a “static texture” is defined as all of the values assumed by a physiological signal at each of the vertices of the surface mesh at a moment t. Similarly, a “dynamic texture” is defined as a temporal series of static textures for a plurality of moments t. FIGS. 6A to 6C in particular show a set 100 of several examples of such static textures. First, FIG. 6A in particular illustrates a static texture of a physiological signal S reconstructed using a method according to the invention, in connection with the brain and the BOLD signal. Likewise, FIG. 6B shows a graphic representation of a static texture of the same original or reference physiological signal S, also in connection with the brain and the BOLD signal. Lastly, FIG. 6C in particular illustrates a static texture of a physiological signal S reconstructed by a method according to the State of the Art, in connection with the brain and the BOLD signal. According to FIG. 6A, in comparison with the experimental BOLD signal described in connection with FIG. 6B and the BOLD signal reconstructed by a method according to the State of the Art, the BOLD signal reconstructed and output by a method according to the invention appears better spatially located and the amplitude of said BOLD signal is clearly better restored than in light of the State of the Art.

Alternatively or additionally, to improve the quality of one or several experimental data or signals obtained and acquired by functional imaging, more specifically by Functional Magnetic Resonance, but also the quality and robustness of the reconstructed physiological signal, a method 200 according to the invention may also comprise one or several prior steps (not shown in the figures) for preprocessing of the experimental datum, said step being arranged to correct said experimental datum.

Indeed, Magnetic Resonance imaging, like all other medical imaging techniques, is not free of artifact formation. Artifacts are observable images not representing any anatomical or physical reality. Quite often, one seeks to avoid or minimize them by modifying certain acquisition or reconstruction parameters. Such artifacts may in fact be of various natures. Furthermore, the Functional Magnetic Resonance imaging acquisition system, more generally functional imaging, may also influence the obtained experimental data. Indeed, the fMRI experimental data, generally in the form of images, result from compromises between any interdependent criteria, such as, but not limited to, the duration of the acquisition, the signal-to-noise ratio, the size of the acquired volume, the spatial resolution or the temporal resolution.

As non-limiting examples, such steps for correcting one or several experimental data may consist of:
a step for correcting movements (realignment), in particular of the head, if the patient does not stay still enough during the acquisition of the sequence, using two successive steps comprising a step for estimating six parameters corresponding to rigid transformations (three translations and three rotations along three axes of the Euclidean space) and a step for transformation of the estimated data using trilinear, sinusoidal or B-spline interpolation methods;
a step for correcting the time offset or intra-slice temporal recalibration correction (also known as “slice-timing”), using a temporal interpolation step, making it possible to consider all slices of a same experimental datum as being acquired at the same moment. Indeed, the acquisition of slices of an experimental datum not being done at the same moment and the duration of said acquisition depending on the repetition time TR, the signals comprised within a same slice may demonstrate a time offset;
an anatomical-functional recalibration step, making it possible to match experimental data (also known as “functional data”) and anatomical data of a subject;
a step for correcting geometric biases and distortions due to non-homogeneities of magnetic fields B1 applied within the Magnetic Resonance imaging apparatus that commonly affect the Magnetic Resonance experimental signals.

Furthermore, as previously specified, the invention provides that a method according to the invention may comprise a prior step (not shown in the figures) for preprocessing of the surface mesh, said step being arranged to recalibrate said surface mesh. Indeed, it may be necessary for the experimental data and the surface mesh to be matched, in order ultimately to reconstruct the physiological signal. Therefore, the surface mesh may advantageously be repositioned in the coordinate system of the experimental datum or data. By way of non-limiting example, the step for recalibrating said surface mesh may comprise one or several recalibration steps similar to those previously described, for example a rigid recalibration step.

The invention further relates to a method 200 for producing a reconstruction of the physiological signal of a region of interest. “Region of interest” refers to any region extending over at least one voxel of interest. Nevertheless, a region of interest cannot be limited to a single voxel, but may include a plurality of voxels, advantageously selected manually or automatically. According to the invention, said physiological signal may be reconstructed in at least two vertices affected by said region of interest for each of said vertices from one or several experimental data using such a method 200 according to the invention, like that previously described, in particular in connection with FIG. 5, said method 200 being implemented by the processing means of the processing unit 4 of a functional imaging analysis system, more particularly Magnetic Resonance imaging, according to FIGS. 1 and/or 2.

Furthermore, as previously specified, in particular in said example described in connection with FIGS. 1 and 2, a functional imaging analysis system according to the invention may comprise output means 5 of the reconstructed physiological signal for a user 6 of said system, said output means 5 cooperating with the processing unit 4. Such output means 5 make it possible to have a rendering, advantageously graphic, audio or the like, and may for example comprise a screen or speakers. Thus, alternatively or additionally, a method 200 according to the invention may advantageously include a subsequent step for triggering the output of the reconstructed physiological signal in one or several vertices of the mesh for each voxel of the region of interest and generating an image from the reconstruction of said physiological signal in the form of a functional activity map in an appropriate format. Such a generation of a functional activity map is implemented owing to a second method consecutive to a method for constructing a physiological signal according to the first object of the invention and is based on methods for example using a general linear model (abbreviated “GLM”). In the context of the preferred exemplary application in connection with the brain, such a functional activity map allows the detection of neuronal activations from the reconstruction of the BOLD signal. FIGS. 8A, 8B and 8C show three examples of functional activity maps generated in the context of the preferred but non-limiting exemplary application, in the case at hand the brain. FIG. 8A illustrates a neuronal activity map generated and output from a BOLD signal reconstructed using a method according to the invention. FIG. 8B in turn illustrates a neuronal activity map from an original or reference BOLD signal. Lastly, FIG. 8C illustrates a neuronal activity map generated and output from a BOLD signal reconstructed using a method according to the State of the Art. According to FIG. 8A, the active zone A of the neuronal activity map clearly appears much more sharply and better obtained than that obtained by a method according to the State of the Art, as shown by comparing FIGS. 8A and 8C in light of the reference coordinate system described by FIG. 8B.

Owing to the new reconstructions of a physiological signal and/or the outputs of said reconstructed physiological signal previously described, the invention makes it possible to provide a user, optionally practitioner, with all relevant and coherent information, information available owing to the use of a method according to the invention. This provision is made possible by an adaptation of the processing unit 4 according to FIG. 1 or 2, in that the processing means of such a processing unit 4 implement a method for reconstructing a physiological signal of a voxel or a region of interest in particular comprising the reconstruction of said physiological signal from one or several experimental data of a voxel of said organ and a surface mesh describing said surface space. Such an implementation is advantageously made possible by the loading or recording, within storage means, optionally comprised within the processing unit 4, cooperating with said processing means, of a computer program product. The latter indeed comprises instructions interpretable and/or executable by said processing means. The interpretation or the execution of said instructions causes or triggers, automatically, the implementation of a method 200 according to the invention. The means for communicating with the outside world of said processing unit can deliver a physiological signal, such as, as a preferred but nonlimiting example, the BOLD signal, in a format appropriate for output means able to output it for a user 6, said reconstructed physiological signal advantageously being able to be output in the form, for example, of time courses, static or dynamic textures, or functional activity maps, such as the examples previously described and illustrated by FIGS. 6A, 7 and 8A. Owing to the invention, the delivered information is more numerous, consistent, reproducible and accurate.

1. A method for reconstructing a physiological signal of an artery/tissue/vein system of an organ in a surface space, said method being implemented by processing means of a processing unit of a functional imaging analysis system, and comprising a step for reconstructing said physiological signal from an experimental datum of a region of interest comprising an elementary volume—called voxel—of said organ and a surface mesh describing said surface space, wherein said step comprises evaluating, according to a method for solving an inverse problem, an a posteriori marginal distribution for said physiological signal in a vertex of said mesh by:
assigning a direct probability distribution of the experimental datum in said surface space based on the parameters involved in the reconstruction problem of the physiological signal of the artery/tissue/vein dynamic system for the voxel in question;
jointly assigning an a priori spatial probability distribution of said physiological signal by introducing a priori information relative to a characteristic of the experimental datum and/or a priori information relative to a property of the artery/tissue/vein dynamic system; and
jointly assigning an a priori temporal probability distribution of said physiological signal by introducing a priori information relative to the impulse response of said artery/tissue/vein dynamic system.

2. A method for reconstructing a physiological signal of an artery/tissue/vein system of an organ in a surface space, said method being implemented by processing means of a processing unit of a functional imaging analysis system, and comprising a step for reconstructing said physiological signal from an experimental datum of a region of interest comprising an elementary volume—called voxel—and a surface mesh describing said surface space, wherein said step comprises evaluating, according to a method for solving an inverse problem, a cost function for said physiological signal in a vertex of said mesh by:
assigning an operator of the direct model establishing the link between the experimental datum in the elementary volume and said physiological signal in said surface space based on the parameters involved in the problem of the reconstruction of the physiological signal of the artery/tissue/vein dynamic system for the voxel in question;
jointly assigning a spatial regularization operator by introducing a priori information relative to a characteristic of the experimental datum and/or a priori information relative to a property of the artery/tissue/vein dynamic system; and
jointly assigning a temporal regularization operator by introducing a priori information relative to the impulse response of said artery/tissue/vein dynamic system.

3. The method according to claim 1, further comprising a step for producing said experimental datum from an acquisition of a signal by functional imaging.

4. The method according to claim 1, the functional imaging analysis system comprising a user interface for the reconstructed physiological signal for a user of said system, said user interface cooperating with the processing unit, said method comprising a subsequent step for triggering a output of the reconstructed physiological signal in an appropriate format.

5. The method according to claim 1, further comprising a prior step for preprocessing of the experimental datum and/or the surface mesh, said step being arranged to correct and/or recalibrate the experimental datum and/or the surface mesh, respectively.

6. The method according to claim 1, when the functional imaging analysis system comprises an interface for a user of said system, said user interface cooperating with the processing unit, further comprising a subsequent step for triggering the output of the reconstructed physiological signal in one or several vertices of the mesh for each voxel of the region of interest and generating an image in the form of a functional activity map.

7. A processing unit of a functional imaging analysis system, said unit comprising an interface for communicating with the outside world and a processor, cooperating with a memory, wherein:
the communication interface is arranged to receive, from the outside world, an experimental datum from an elementary volume of an organ,
the memory contains instructions executable or interpretable by the processor, whereof the interpretation or execution of said instructions by said processor causes the implementation of a method according to claim 1.

8. The processing unit according to claim 1, wherein the communication interface delivers a reconstructed physiological signal in an appropriate format to an interface suitable for retrieving it for a user.

9. A functional imaging analysis system comprising a processing unit according to claim 7 and an interface configured to output, for a user, a physiological signal using said method implemented by said processing unit.

10. A non-transitory computer-readable medium containing a computer program comprising one or several instructions interpretable or executable by the processor of a processing unit according to claim 7, said processor cooperating with a memory, said program being loadable in said memory, wherein the interpretation or execution of said instructions by said processor causes the implementation of said method.

11. The method according to claim 2, further comprising a step for producing said experimental datum from an acquisition of a signal by functional imaging.

12. The method according to claim 2, the functional imaging analysis system comprising a user interface for the reconstructed physiological signal for a user of said system, said user interface cooperating with the processing unit, said method comprising a subsequent step for triggering a output of the reconstructed physiological signal in an appropriate format.

13. The method according to claim 2, further comprising a prior step for preprocessing of the experimental datum and/or the surface mesh, said step being arranged to correct and/or recalibrate the experimental datum and/or the surface mesh, respectively.

14. The method according to claim 2, when the functional imaging analysis system comprises an interface for a user of said system, said user interface cooperating with the processing unit, further comprising a subsequent step for triggering the output of the reconstructed physiological signal in one or several vertices of the mesh for each voxel of the region of interest and generating an image in the form of a functional activity map.

15. A processing unit of a functional imaging analysis system, said unit comprising an interface for communicating with the outside world and a processor, cooperating with a memory, wherein:
the communication interface is arranged to receive, from the outside world, an experimental datum from an elementary volume of an organ,
the memory contains instructions executable or interpretable by the processor, whereof the interpretation or execution of said instructions by said processor causes the implementation of a method according to claim 2.

16. The processing unit according to claim 2, wherein the communication interface delivers a reconstructed physiological signal in an appropriate format to an interface suitable for retrieving it for a user.

17. A functional imaging analysis system comprising a processing unit according to claim 15 and an interface configured to output, for a user, a physiological signal using said method implemented by said processing unit.

18. A non-transitory computer-readable medium containing a computer program comprising one or several instructions interpretable or executable by the processor of a processing unit according to claim 15, said processor cooperating with a memory, said program being loadable in said memory, wherein the interpretation or execution of said instructions by said processor causes the implementation of said method.
